(19) United States
(12) Patent Application Publication
(10) Pub. No.: US 2020/0179362 A1
(43) Pub. Date: Jun. 11, 2020

(54) DIETARY SUPPLEMENT COMPRISING GLUCURONIDATION INHIBITION ASSIST FOR HEALTHFUL DAILY SYNERGY OF ANTI-INFLAMMATION, CELLULAR ANTI-OXIDANT BOOST, CELLULAR PROTEIN CLEAN-UP BOOST

(71) Applicant: James E. Beecham, West Palm Beach, FL (US)

(72) Inventor: James E. Beecham, West Palm Beach, FL (US)

(21) Appl. No.: 16/546,919

(22) Filed: Aug. 21, 2019

Related U.S. Application Data
(63) Continuation-in-part of application No. 16/210,890, filed on Dec. 5, 2018.
Application No. 16/210,890 is a continuation-in-part of application No. 16/389,969, filed on Apr. 21, 2019.
(60) Provisional application No. 62/875,028, filed on Jul. 17, 2019.
(60) Provisional application No. 62/877,111, filed on Jul. 22, 2019.

(51) Int. Cl. A61K 31/4525; A61K 31/05; A61K 31/197; A61K 31/12; A61K 31/155; A61K 31/4415; A61K 33/06; A61K 36/74

(52) U.S. Cl. CPC A61K 31/4525; A61K 31/05; A61K 31/197; A61K 36/74; A61K 31/155; A61K 31/4415; A61K 33/06; A61K 31/12

(57) ABSTRACT

The present invention comprises a method and system for configuring a dietary supplement and providing same to user, linked to publically accessible notice at least referencing partial autophagy effect. Said dietary supplement can be configured as tablet, capsule, liquid aliquot, powder, pill, food additive or other common or uncommon forms of dietary supplement. Said dietary supplement comprises in recommended daily dose over years, as a majority of milligrams of active ingredients: at least one component to reduce inflammation, at least one component to boost local cellular production of antioxidants, at least one component to boost local cellular clean-up of damaged protein, at least one component to inhibit glucuronidation.

### CROSS-REFERENCE TO RELATED APPLICATIONS

The present application claims priority to U.S. patent application Ser. No. 16/210,890, filed on Dec. 5, 2018 entitled “Method and system for preserving cognitive capacity during aging”, U.S. patent application Ser. No. 16/389,969, filed on Apr. 21, 2019 entitled “Method and system for preserving cognitive capacity during aging”, U.S. Provisional Application Ser. No. 62/875,028 filed Jul. 17, 2019, and U.S. Provisional Application Ser. No. 62/877,111 filed Jul. 22, 2019, the disclosures of which are hereby incorporated in their entirety at least by reference.

### BACKGROUND OF THE INVENTION

### 1. Field of the Invention

The present invention generally relates to dietary supplements but more particularly to convenient dietary supplement comprising glucuronidation inhibition assist for healthful daily synergy of contemporaneous anti-inflammation, cellular antioxidant boost, and cellular protein clean-up boost.

### 2. Description of Related Art

Dietary supplements to improve health are well known. Nevertheless, the health of many supplement users still suffers. Various dietary supplements have met with limited success

### BRIEF SUMMARY OF THE INVENTION

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

In one aspect of the invention, a dietary composition is provided, comprising: a synergistic mixture of ingredients including piperine, lutein, ornithine, and curcumin; the synergistic mixture of ingredients configured to conveniently, contemporaneously and beneficially reduce inflammation, enable boosting of level of antioxidant, and enable boosting of partial autophagy when consumed by a user.

In another aspect of the invention, a convenient method of increasing cellular protection by contemporaneously reducing inflammation, enabling boosting of level of antioxidant, enabling boosting of partial autophagy in a mammal in need thereof is provided, the method comprising: administering to the mammal a therapeutically effective amount of a dietary composition comprising a synergistic mixture of ingredients including piperine, lutein, ornithine, and curcumin.

In another aspect of the invention, a method for preventing a disease or disorder in a subject in need thereof, wherein said disease or disorder is associated with excess inflammation, dis-enabled antioxidant production, dis-enabled partial autophagy is provided, the method comprising: administering to the subject a therapeutically effective amount of a dietary composition comprising a synergistic mixture of ingredients including piperine, lutein, ornithine, and curcumin.

In yet another aspect of the invention, a dietary supplement is provided, consisting of lutein, ornithine, curcumin, glucuronidation inhibitor, and at least one of: agmatine, at least one form of vitamin B6 such as pyridoxal 5¹ phosphate, manganese, taurine, a combination of chlorogenic acid and caffeine as green coffee bean extract.

The foregoing has outlined rather broadly the more pertinent and important features of the present disclosure so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present disclosure. It should be realized by those skilled in the art that such equivalent structures do not depart from the spirit and scope of the invention as set forth in the appended claims.

### DETAILED DESCRIPTION OF THE PREFERRED EMBODIMENT

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein to specifically provide convenient dietary supplement comprising glucuronidation inhibition assist for healthful daily synergy of antiinflammation, cellular antioxidant boost, and cellular protein clean-up boost.

As used in this application, the term “a” or “an” means “at least one” or “one or more.” As used in this application, the term “about” or “approximately” refers to a range of values within plus or minus 10% of the specified number. As used in this application, the term “convenient” refers to a dietary supplement which is of a composition conducive to relatively quick and easy consuming. As used in this application, the term “substantially” means that the actual value is within about 10% of the actual desired value, particularly within about 5.% of the actual desired value and especially within about 1% of the actual desired value of any variable, element or limit set forth herein. Any reference to “or” herein is intended to encompass “and/or” unless otherwise stated. As used herein, the term “about” refers to an amount that is near the stated amount by about 0%, 5%, or 10%, including increments therein. The word “synergistic” is defined to mean the combined effect of two or more ingredients in the composition. As used herein, the term “configure” refers to the formulating together of ingredients within a convenient dietary supplement.

In one embodiment, the instant dietary supplement comprises at least one active ingredient comprising:

a.) at least one component suitable when consumed to limit inflammation;

b.) at least one component suitable when consumed to boost local cellular production of antioxidants;

c.) at least one component suitable when consumed to boost local cellular clean-up of damaged protein; and,

d.) at least one component suitable when consumed to inhibit glucuronidation.

The active ingredients combination(s) described throughout this disclosure, where stated to comprise at least a majority of the active ingredient milligrams, are those where such active ingredients in milligrams represent at least half or more of the total of active ingredients' milligrams within the respective disclosed instant invention composition.

In one embodiment, the instant dietary supplement comprises as at least one active ingredient comprising:

a.) a lutein;

b.) a curcumin;

c.) an ornithine; and

d.) a piperine-based inhibitor of glucuronidation.

It is well known that disease is often the result of multiple simultaneous factors operating to produce damage to health within a human body contemporaneously, and for extended number of days. For example, excess and long-term chronic inflammation can contribute to cellular damage and organ dysfunction, especially where natural production of antioxidants is contemporaneously low, and especially where natural ‘clean-up’ of damaged intra-cellular protein is also contemporaneously deficient.

Natural compounds shown to reduce inflammation when consumed include curcumin. Curcumin is found in curried foods. It can be reasonably argued that most people are relatively deficient in dietary curcumin.

Further, it is well known that an antioxidant produced locally, at the site of oxidation risk within a cell, is a thousand times more effective than an orally administered antioxidant, such as oral vitamin C. Natural compounds shown to boost local natural production of antioxidants such as glutathione include nrf2 activators such as lutein, a natural yellow xanthine found in some fruits and vegetables. It can be reasonably argued that most people are relatively deficient in natural nrf2 activators, such as lutein, and hence likely at increased risk for excess cellular oxidative damage.

Yet additionally, it is well known that damaged protein within a cell contributes to cell dysfunction, especially when such damaged intra-cellular protein accumulates and binds metal ions such as magnesium, zinc or copper, thence to act as a promotion nidus for oxidation damage. Because oxidation damage within a cell is a self-propagating process, the ‘tinder’ of intra-cellular damaged protein can allow the ‘fire’ of damaging oxidation to proceed more rapidly, thus damaging the cell internally.

Alternatively, removing daily any excess build-up of damaged protein within a biologic cell is a form of natural protective ‘clearing away of the dry brush’, especially useful as daily preventative against the ‘fire’ of oxidation damage arriving or propagating within the cell. The natural system provided by evolution to accomplish such ‘clearing away’ is known as partial autophagy.

Partial autophagy requires a signal to execute such beneficial clearing away of damaged protein, such clearing typically operating via action of chaperone proteins and lysosomes. To activate such partial autophagy factor, typically spermidine is needed to act within the cell. However, the level of spermidine in a cell is typically or intermittently very low, often too low for optimal health. Spermidine can be supplied by wise choices in diet, or by endogenous production of spermidine via the polyamine pathways, provided precursors and enzymes and enzyme co-factors of those pathways are provided.

Dietary sources of spermidine-rich foods include several foods which most people typically find relatively unappealing, such as liver and certain fermented vegetables. Beyond diet sources of pre-formed spermidine, local enzymatic conversion of polyamine pathway amino acids which can add to spermidine for activation of partial autophagy, is often inadequate due to lack of needed precursor amino acids or lack of enzyme or enzyme co-factor, or a combination of these issues.

Polyamine precursors for spermidine include ornithine and agmatine. The conversion of these precursor amino acids into next step (putrescene) in pathway requires the local presence of adequate amounts of specific enzymes to enable the said conversion. Deficiencies of such enzymes and/or their co-factors are common. These deficiencies can be at least partially overcome by wise diet choices and/or by supplying needed substances in supplement form. Nevertheless, it can be reasonably argued that most modern people are at least intermittently deficient in spermidine, for one or more of the reasons above enumerated, and hence deficient in beneficial partial autophagy activity in cell interiors.

Thus, most people suffer from inadequate clean-up of damaged cell-internal protein due to lack of activation of partial autophagy. In such circumstance, where abnormal protein accumulates within a cell, and where an important enzyme needs a metal ion as co-factor, the metal ion transit to engage the enzyme as co-factor is less likely to occur because the metal ion can become bound instead by the accumulated protein. Thus, most modern people are at risk for excess oxidation inside cells from nidus effect of damaged protein bound to metal ions, and most modern people are simultaneously at risk for low co-factor activity of co-factor metal ions needed to assist key enzymes.

Additionally, it is well known that piperine, the alkaloid responsible for the pungency of black pepper, can be extracted from black pepper, optionally chemically modified, and thus able to serve within dietary supplements, when consumed, to degrade the activity of glucuronidation. Glucuronidation is a natural process typically carried out by certain CYP enzymes of the liver, and by which the formation of water-soluble substrates from xenobiotics leads to such xenobiotics being relatively rapidly eliminated from the body in bile or urine. While the action of these CYP enzymes for purposes of glucuronidation are typically beneficial, the same process can remove beneficial compounds from the body rapidly. Slowing that removal of beneficial compounds is a known application for piperine and piperine-based compounds within dietary supplements.

The results of clinical studies suggest that piperine (usually in doses of 20 mg/day in the study) can inhibit CYP3A4, CYP2C9, and PGP. By inhibiting such glucuronidation enzymes, the piperine-style compounds, such as piperine, Bioperine® and the like, can preserve activity in the body of certain other natural compounds, such as from diet or from a consumed supplement. In this manner, piperine-style compounds can protect these beneficial compounds from glucuronidation, thereby prolonging their bioavailability to accomplish longer their beneficial purposes. It can be reasonably argued that many people are relatively deficient in natural inhibitors of glucuronidation, and furthermore that many such people thereby suffer limited activity in their bodies of key natural substances otherwise suitable to reduce inflammation, to boost natural antioxidant production, or to boost natural partial autophagy activity.

It is scientifically viable to state that, as a result of daily consumption over years by user of instant dietary supplement, a user in need can be expected to be more likely to experience synergistic multiple beneficial actions in their bodies, to comprise:
a.) Anti-inflammatory activity;
b.) Local production of antioxidants to limit damage from oxidative stress;
c.) Local activation of partial autophagy in cells to clear damaged protein; and,
d.) Prolongation of at least one of above actions via activity of inhibitor of glucuronidation.

In one embodiment, instant invention dietary supplement comprises as majority of active ingredient milligrams, two or more of: piperine, lutein, agmatine, ornithine, curcumin, pyridoxal 5¹ phosphate (PLP) taurine, caffeine, chlorogenic acid and manganese. The scientific logic of including vitamin B6 PLP is that PLP is co-factor to ornithine decarboxylase, and thus PLP assists in conversion by enzyme of omithine to putrescene. The scientific logic of including manganese is that manganese is co-factor to agmatinase, and thus manganese assists in conversion by enzyme of agmatine to putrescene. By boosting putrescene levels, the likelihood of polyamine pathway downstream production of spermidine from putrescene is increased. Thus, the likelihood increases that beneficial partial autophagy will occur for users in need to clean away damaged protein from the interior of the cell, reducing the chance of cellular damage by oxidation. Typically, PFP is present in instant invention dietary supplement at level for adult as 1 milligram per daily dose. Typically, manganese is present in instant invention dietary supplement at level for adult as 1.2 milligrams per daily dose.

In one embodiment, the instant invention dietary supplement is advised via linked notice to user to be consumed daily and for years. Optionally such notice explains that early use is especially beneficial in adult life, i.e. prior to disease onset in the user. This advised schedule of use beginning early in adult life and daily use for years is based on science, as herein further explained. Typically, the notice at least references at least one aspect related to partial autophagy.

To understand why such use is beneficial, it is useful to consider how enzyme co-factors locate and activate enzymes in the body. Such locating is done via random movement of co-factors within cell interiors, until such co-factor bumps into and links with the corresponding enzyme needing activating. But such bumping into in timely fashion requires ease of access within the cell interior. Such ease of access is impeded when the cell interior is cluttered, such as by oxidized built-up damaged protein which can bind the co-factor or otherwise impede it.

By contrast, a normal and uncluttered interior makes it easier for co-factors, even in micro-molar amounts, to find and link with enzymes. This normal state is advantageous because it keeps user's cells free from over-load by oxidized abnormal protein, and it keeps uncluttered the cell interiors to enable free transit of co-factors to meet and activate enzymes.

The uncluttered cell interior allows dietary ingredients such as enzyme co-factors to find, within the uncluttered cell interiors, the enzymes for which they should act as co-factor. Thus, a small amount of dietary or supplemented vitamin B6 PLP, for example, can provide co-factor action to ornithine decarboxylase (ODC). Thus, a small amount of dietary or supplemented manganese ion can provide co-factor action for agmatinase.

By contrast, in a cell overloaded with oxidized damaged protein, transit of vitamin B6 to locate and activate ODC is impeded. For users with already cluttered cell interiors, the relatively normal amount in diet or supplement of B6 or manganese will likely not be effective. For example, a normal small amount of manganese in diet or in a supplement cannot, within a cluttered cell, transit to reach and activate agmatinase, because such transit is impeded by clutter.

The diet becomes deficient because the cell is cluttered on its interior. Then, even when supplied with normal amounts of co-factors, the cell cannot experience partial autophagy or antioxidation because the clutter prevents co-factor activation of enzymes due to impediments. From this analysis it is clear why daily application of instant invention dietary supplement is important to take place early in adult life, and to supplement diet daily on schedule over years, to make sure that uncluttered cell interiors are preserved. In cluttered cell interiors, often the metal ions are bound to damaged protein, thus reducing the availability of metal ions to serve as co-factors to enzymes or the like.

Consumption of the instant invention dietary supplement early in adult life, and regularly as daily with each meal, and for years is the typical schedule advised on linked notice to user, optionally also accessible notice by public. By such consuming daily for years and before clutter has set in, it is thus possible for small amounts of co-factors, either in diet or in supplement, to continue to activate protective actions to prevent interior clutter and damage.

By such preventing of disorder/disease, the user will more likely continue to experience health. Such protective measures include antioxidant production locally in a cell, reduction of excess inflammation, and spermidine production via polyamine pathways to activate partial autophagy to maintain user cells in non-cluttered state.

It can thus be seen why use of the instant invention dietary supplement is advised, typically via a supplement-linked notice to user, as consumed with each meal and regularly for weeks, months and years, so as to be prior to advent of over-loaded and impeded cell-internal state, and to keep such state from developing. In this way, a disease or disorder can be prevented or delayed or reduced, especially a disorder or disease related in causation to excess inflammation, excess damaging oxidation of cell parts and/or excess build-up of damaged cell protein.

In a preferred embodiment, the present invention comprises a system and method for providing a user with daily dose of dietary supplements, each of which typically comprises, as a majority by milligram weight of its active ingredients: lutein, curcumin, at least one inhibitor of glucuronidation, and at least one of: ornithine or agmatine.

In a preferred embodiment, the dietary supplement of the present invention comprises milligram amounts of the said ingredients which are within levels found safe in published clinical study and/or are at a level in the dietary supplement daily dose so as to be similar in amount to that achievable daily by healthful diet.

In a preferred embodiment, the said dietary supplement further comprises a linked publicly accessible notice wherein health is at least referenced. The notice optionally at least references benefit regarding at least one of: antiinflammation, antioaidation and protein clean-up.

In one embodiment, lutein, a yellow xanthophyll carotenoid found in egg yolks and many fruits and vegetables, most notably leafy green vegetables like kale and spinach is accessed for amount in dietary supplement versus amount of lutein known to be present in average modern Western diet at only 1 mg to 2 mg per day, an inadequate amount for full health. By contrast, in the assessment of amount of lutein to include in dietary supplement daily dose herein, it is noted that published medical studies of amount of daily lutein needed to protect against age-related macular degeneration suggests 6 mg per day is an appropriate daily protective amount of lutein to consume.

Curcumin, a biologically active polyphenolic compound found in turmeric, is a common spice derived from the rhizomes of the plant Curcuma longa Linn. Curcumin has been used safely in the range 4 grams per day to 8 grams per day in clinical studies. The anti-inflammatory effects of curcumin result from its ability to inhibit the NF-κB pathway, as well as other pro-inflammatory pathways like the mitogen-activated protein kinase (MAPK) and the Janus kinase (JAK)/Signal transducer and activator of transcription (STAT)-dependent signaling pathways. To safely apply such anti-inflammatory action of curcumin, a dose closer to the study level versus daily inadequate diet level is preferred herein. The average consumption of curcumin in diet can be from nil to just an occasional curry food dish with a few milligrams.

Regarding ornithine, fifty-two apparently healthy Japanese adults who had previously felt slight stress as well as fatigue were recruited to be study participants and were randomly divided into either the L-ornithine (400 mg/day) or placebo group. They orally consumed the respective test substance every day for 8 weeks. No adverse effects were noted, and researchers concluded that L-omithine supplementation has the potential to relieve stress and improve sleep quality related to fatigue, both objectively and subjectively. By contrast, the foods typically consumed by an average person are low in ornithine content.

Regarding agmatine, a clinical study was published describing two human subjects each consuming daily dose of 2.67 g agmatine sulfate, encapsulated in gelatin capsules, six capsules daily, each containing 445 mg, three in the morning and three in the evening after meals, for 5 years without adverse effect. In studies of use of agmatine sulfate in diabetic patients suffering from neuropathic pain, agmatine sulfate had a significant effect in decreasing burning, tingling and unpleasant aspects of neuropathic pain at a dose, during a two-month treatment period, of agmatine sulfate 3.670 g/day. In diet, agmatine is highest in fermented foods and fermented beverages, and also in protein-rich foods, e.g., fish and fish products, meat and meat products, eggs, cheeses, fermented vegetables, fruits, nuts, chocolate, soybean products, and wine.

Daily diet amount of agmatine are not well established, but it appears in general that an average human diet provides only a few milligrams but with wide variability. For example, agmatine concentration has been reported to vary from not detected to 22 mg/L in red wines and from not detected to 6.5 mg/L in white wines.

Regarding piperine, the major bio-active component of pepper, which imparts pungency and biting taste to it, this naturally occurring alkaloid has numerous demonstrated health effects and beneficial therapeutic properties; nevertheless, its biological applications are limited due to its poor solubility in aqueous environments. The amount of piperine varies in plants belonging to the Piperacwe family, it constitutes 2% to 7.4% of both black pepper and white pepper.

Taurine, an amino sulfonic acid (also known as a conditionally essential amino acid or beta amino acid), has been found to be an effective therapy against congestive heart failure. Taurine has also been found to be supportive in treatment of several other diseases including inflammatory diseases such as arthritis and stroke-like episodes. The main dietary sources of taurine are animal foods, such as meat, fish and dairy. Smaller amounts occur in some plant foods. An average diet provides about 40-400 mg of taurine per day. The cytoprotective actions of taurine contribute to the improvement in the clinical and nutritional health of humans through various mechanisms, including antioxidation, energy production, neuromodulation, Ca2+ homeostasis and osmoregulation. In a randomized clinical trial involving patients with chronic liver disease, 2 grams per day of supplemental taurine was administered without adverse side effects. The instant invention dietary supplement daily dose, for those supplements which comprise taurine, have that taurine in amount typically of 150 milligrams of taurine in a daily dose. Within the various formulations of the dietary supplements within the instant invention, those dietary supplements which comprise taurine have that taurine in amount in daily dose typically within a range of 10 milligrams to 3 grams per daily dose.

Piperine has immunomodulatory, anticarcinogenic, stimulatory, hepatoprotective, anti-inflammatory, antimicrobial, and antiulcer activities. Piperine has bio-transformative effects and can enhance the bioavailability of different drugs, apparently via inhibiting glucuronidation of other substances in the body.

The pharmaceutical activities of piperine are limited due to its low water solubility and because use of it at high concentrations can be toxic for the central nervous and reproductive systems. Attempts have been made to develop new formulations to improve the biosvailability of piperine, such as via use of modern nanotechnology for nanoparticle formulations of piperine and its encapsulation in lipid matrixes to overcome its low solubility in water. Piperine can enhance or reduce the biologic activity of certain drugs. The absorption of piperine across the intestinal barrier is very fast. The results of clinical studies suggest that piperine (usually in doses of 20 mg/day) can inhibit CYP3A4, CYP2C9, and PGP. Although pepper is a commonly used spice, the average dietary intake of piperine is estimated for most humans to be 1 milligram daily approximately.

To summarize, the study dose of selected ingredient substances mentioned herein, and their typical amount available from average diet are:

a.) Lutein—diet 1 mg daily; study 6 mg daily;

b.) Curcumin—diet—curried food/0.01 gram daily; clinical study 8 grams daily;

c.) Piperine—diet −1 milligrams per day (variable)—study −10 milligrams daily;

d.) Ornithine—diet—liver/fermented food—variable 0.01 grams daily; study 0.4 g daily;

e.) Agmatine—diet—2 milligrams per day (variable)—3.67 g daily; and\

f.) Taurine—diet—40-400 milligrams per day—study up to 2 grams daily.

To summarize, clinical studies support judging the effective amount in daily dose of selected ingredients cited herein as: piperine at least 1 mg, lutein at least 1 mg, ornithine at least 10 mg, curcumin at least 10 mg.

A variety of configurations of instant invention dietary supplement are contemplated. Said dietary supplement can be of any of standard modes, such as tablet, capsule, liquid aliquot, powder, pill, food additive or other common or uncommon known forms of dietary supplement.

For example, the ingredients for a daily dose can be from multiple such aliquots which taken, for example tablets with meals, wherein such tablets taken as recommended over a day will total a daily dose. For example, daily doses can be of a variety of formulations, such as said dietary supplement daily dose comprising ingredients in following amount ranges as to recommended daily dose: lutein in amount range of 1 mg per day to 6 mg per day; curcumin 0.01 gram daily to 8 grams daily; ornithine 0.01 grams daily to 0.8 grams daily; agmatine 0.02 milligrams daily to 3 grams daily, piperine 0.1 milligram daily to 10 milligrams daily. In one embodiment, a tablet is comprised as majority of active ingredient milligrams to comprise: lutein 2 mg curcumin 100 mg taurine 100 mg ornithine 30 mg, agmatine 20 mg and piperine 3 mg. Such tablet is recommended to user as one tablet per meal, at each of three meals in a day, and similar to be consumed for months and years. Thus, the daily dose comprises 3 said tablets, for a total daily of lutein 6 mg, curcumin 300 mg, taurine 300 mg, ornithine 90 mg, agmatine 60 mg and piperine 9 mg.

In one embodiment of the instant invention dietary supplement, a supplement comprises curcumin, lutein, ornithine, piperine, chlorogenic acid and caffeine as extract of green coffee beans. Green coffee beans are coffee seeds (beans) of Coffea fruits that have not yet been roasted. The roasting process of coffee beans reduces amounts of the chemical chlorogenic acid. Therefore, green coffee beans have a higher level of chlorogenic acid compared to regular, roasted coffee beans. Chlorogenic acid in green coffee is thought to have health benefits. Chlorogenic acid benefits in weight loss, are thought to derive from how chlorogenic acid affects how the body handles blood sugar and metabolism. Caffeine is a central nervous system stimulant of the methylxanthine class. Caffeine is the world's most widely consumed psychoactive drug. The green coffee bean extract, when taken in moderate amount by human user, has been shown in research to have a pharmacologic profile comprising modest weight loss in obese individual users, and mild effects of caffeine. Caffeine's well-known biologic effects in human users typically comprise a boost in antioxidants such as glutathione and a positive effect on cognitive performance such as improvement in delayed word recognition reaction time, improved Rapid Visual Information Processing (RVIP) accuracy and reduced self-reported ‘mental fatigue’. The typical amount of green coffee bean extract (GCBE) in dietary supplements hereinunder comprising chlorogenic acid and caffeine is 50 milligrams GCBE per daily dose.

In one embodiment, the instant invention dietary supplement is recommended to user for consumption daily over years. In this manner, the action of the inhibitor of glucuronidation is more potent over time, due to the delay of the body to produce additional CYP enzymes after piperine-based compound reduces the action of glucuronidation in the body. In this manner, the co-consumptions herein disclosed via daily use over years of the instant invention dietary supplement formulations are enabled to benefit health of user in a progressively stronger manner over initial days to weeks.

In one embodiment, instant invention dietary supplement comprises as a majority of active ingredients comprising: piperine, lutein, agmatine, ornithine, curcumin, pyridoxal 5^{I} phosphate (PLP), and manganese. The scientific logic of including vitamin B6 PLP is that PLP is co-factor to ornithine decarboxylase, and thus PLP assists in conversion by enzyme of ornithine to putrescine. The scientific logic of including manganese is that manganese is co-factor to agmatinase, and thus manganese assists in conversion by enzyme of agmatine to putrescene. By boosting putrescene levels, the likelihood of polyamine pathway downstream production of spermidine from putrescene is increased. Thus, the likelihood increases that beneficial partial autophagy will occur to clean away damaged protein, reducing the chance of cellular damage by oxidation. Typically, PFP is present in instant invention dietary supplement at level for adult as 1 milligram per daily dose. Typically, manganese is present in instant invention dietary supplement at level for adult as 1.2 milligrams per daily dose.

In one embodiment, the instant invention dietary supplement is advised via linked notice to user to be consumed daily and for years. Optionally such notice explains that early use is especially beneficial in adult life, i.e. prior to disease onset in the user. This advised schedule of use beginning early in adult life and daily use for years is based on science, as herein further explained.

To understand why such use is beneficial, it is useful to consider how enzyme co-factors locate and activate enzymes in the body. Such locating is done via random movement of co-factors within cell interiors, until such co-factor bumps into the enzyme needing activating But such bumping into in timely fashion requires ease of access within the cell interior. Such ease of access is impeded when the cell interior is cluttered, such as by oxidized built-up damaged protein.

By contrast, a normal and uncluttered interior makes it easy for co-factors, even in micro-molar amounts, to find and interact with enzymes. This normal state is advantageous because it keeps user's cells free from over-load by oxidized abnormal protein and opens the cell interior to transit of co-factors to meet and activate enzymes.

The uncluttered cell interior allows dietary ingredients such as enzyme co-factors to find, within the uncluttered cell interiors, the enzymes for which they should act as co-factor. Thus, a small amount of dietary or supplemented vitamin B6, for example, can provide co-factor action to ornithine decarboxylase (ODC). Thus, a small amount of dietary or supplemented manganese ion can provide co-factor action for agmatinase, the manganese metalloenzyme of the ureohydrolase superfamily which serves to convert agmatine to putrescene.

By contrast, in a cell overloaded with oxidized damaged protein, transit of vitamin B6 to locate and activate ODC is impeded. For users with already cluttered cell interiors, the relatively normal amount in diet or supplement of B6 or manganese will not be effective. For example, a normal small amount of manganese in diet or in a supplement cannot, within a cluttered cell, transit to reach and activate agmatinase, because such transit is impeded by clutter.

The diet becomes deficient because the cell is cluttered on its interior. Then, even when supplied with normal amounts of co-factors, the cell cannot experience partial autophagy or antioxidation because the clutter prevents co-factor activation due to impediments. From this analysis it is clear why daily application of instant invention dietary supplement is important to take place early in adult life, and to supplement diet to make sure that uncluttered cell interiors are preserved.

Consumption of the instant invention dietary supplement early in adult life, and regularly as daily with each meal, and for years is the typical schedule advised on linked notice to user, optionally also accessible notice by public. By such consuming daily for years and before clutter has set in, it is thus possible for small amounts of co-factors, either in diet or in supplement, to continue to activate protective actions to prevent interior clutter.

By such preventing, the user will be more likely to continue to experience health. Such protective measures include antioxidant production locally in a cell, reduction of excess inflammation, and spermidine production via polyamine pathways to activate partial autophagy to maintain user cells in non-cluttered state.

It can thus be seen why use of the instant invention dietary supplement is advised, typically via a supplement-linked notice to user, as consumed with each meal and regularly for years, so as to be prior to advent of over-loaded and impeded state, and to keep such state from developing.

In one embodiment, the instant invention comprises as daily dose of dietary supplements, five tablets consisting of two of tablet A in a day, taken one at breakfast and one at lunch, and two of tablet B in a day, taken half-tablet at breakfast, half-tablet at lunch and full tablet at dinner, and wherein tablet A comprises green coffee bean extract 25 mg, taurine 50 mg, agmatine 50 mg, pyridoxal 5ⁱ phosphate 1 mg, manganese 0.5 mg; and tablet B comprises 90 mg curcumin, 30 mg L-ornithine, 4 mg lutein, and 3 milligram piperine. This combination of tablet A and tablet B, consumed by user per stated schedule, is new, non-obvious and useful, at least because, for user.
1. It is convenient—user time is saved compared to dosing with 9 separate ingredients
2. It supplies beneficial effects timed to match biologic needs, as described herein, to include:
   a. the delivery of the ingredient caffeine at breakfast and lunch (not dinner) so that the caffeine's stimulatory effects of more efficient wakeful brain activity occurs during the daytime, and does not disturb sleep pattern, and
   b. the timed antioxidant production boost (TAPB) associated to ingredients lutein, curcumin, taurine and caffeine causes increased cell-internal levels of natural antioxidants (such as glutathione) at beneficial timing i.e. coordinated to times when meal intake of food calories daily causes daily spikes in oxidation damage potential in the body due to processing of mealtime consumed food calories, and
   c. the boost to beneficial partial autophagy activity of ingredients working together to reduce build-up of cell-internal damaged protein debris, as amino acids from consumed food are used in cell interiors (at ribosomes) to construct protein, a process which is subject to abnormal protein production and build-up. Such coordination of partial autophagy boost (CPAB) occurs via cell-internal polyamine pathways enabled by ingredients of the Tablet A plus Tablet B combination, i.e. ornithine, agmatine to produce putrescene and hence spermidine, as earlier explained, plus further enabled by ingredients used as enzyme co-factors in the polyamine pathway, i.e. pyridoxal 5¹ phosphate and manganese, and these proceed apace with consumed food in digestion, and
   d. the beneficial calming of chronic inflammation (BCCI) which chronic inflammation otherwise rises from toxins in mealtime food intake. Such BCCI is accomplished at least by anti-inflammatory ingredient curcumin, and
   e. the glucuronidation inhibition supplied by ingredient piperine, whereby the duration of the combined and timed beneficial effects of at least one of the other ingredients is prolonged, such as for TAPB and CPAB and BCCI.

Among potential arguments incorrectly asserted against patentability of the instant invention could be a.) an obviousness as to prior art; and b.) a likelihood of a person of average skill to perform this invention from prior disclosures. However, the recent decision of the Federal Circuit is on point as to why such arguments are considered by instant inventor to be incorrect as regards the instant invention. In the absence of the knowledge herein disclosed, a person of average skill in the art is highly unlikely to accidentally stumble on the co-consumptions herein disclosed, especially as to the convenient configuring and the timed consuming of the herein disclosed formulations daily and for years. For example, the daily consuming for years of the dietary supplement formula herein above disclosed in a convenient embodiment comprising curcumin plus the two polyamine precursors ornithine and agmatine, plus the two enzyme co-factors manganese and pyridoxal 5l phosphate, plus taurine, green coffee bean extract, piperine and lutein, is judged unlikely to be stumbled upon.

Although the invention has been described in considerable detail in language specific to structural features, it is to be understood that the invention defined in the appended claims is not necessarily limited to the specific features described. Rather, the specific features are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated and can be made without departing from the spirit and scope of the invention.

In addition, reference to “first,” “second,” “third”, etc. members throughout the disclosure, and in particular the claims, are not used to show a serial or numerical limitation but instead are used to distinguish or identify the various members of the group.

What is claimed is:

1. A dietary composition comprising:
a synergistic mixture of ingredients including piperine, lutein, ornithine, and curcumin;
the synergistic mixture of ingredients configured to contemporaneously reduce inflammation, enable boosting of cellular level of antioxidant, and enable boosting of partial autophagy when consumed by a user.

2. The dietary composition of claim 1, further comprising at least one additional ingredient of: agmatine, vitamin B6, manganese, taurine, and green coffee bean extract.

3. The dietary composition of claim 1, configured for convenient consumption and advised to be consumed by mammal with meals.

4. The dietary composition of claim 2 linked to a notice at least referencing at least one aspect of partial autophagy.

5. A method of increasing cellular protection by contemporaneously reducing inflammation, enabling boosting of level of antioxidant, enabling boosting of partial autophagy in a mammal in need thereof, the method comprising:
administering to a mammal a therapeutically effective amount of a dietary composition comprising a synergistic mixture of ingredients including piperine, lutein, ornithine, and curcumin; wherein the effective amount in dietary composition comprises at least 1 mg of the piperine, at least 1 mg of lutein, at least 10 mg of omithine, and at least 10 mg of the curcumin.

6. The method of claim 5, wherein the dietary composition further comprises at least one additional ingredient of: agmatine, vitamin B6, manganese, taurine, and green coffee bean extract.

7. A method for preventing a disease or disorder in a subject in need thereof, wherein said disease or disorder is associated with excess inflammation, dis-enabled antioxidant production, dis-enabled partial autophagy, the method comprising:
administering to the subject a therapeutically effective amount of a dietary composition comprising a synergistic mixture of ingredients including piperine, lutein, ornithine, and curcumin; wherein the effective amount in dietary composition comprises at least 1 mg of the piperine, at least 1 mg of lutein, at least 10 mg of ornithine, and at least 10 mg of curcumin.

8. The method of claim 7, wherein the dietary composition further comprises at least one additional ingredient of: agmatine, vitamin B6, manganese, taurine, and green coffee bean extract.

9. A dietary supplement consisting of:
lutein, ornithine, curcumin, glucuronidation inhibitor, and at least one of: agmatine, vitamin B6, manganese, taurine, and green coffee bean extract.

10. The dietary supplement of claim 9, wherein the dietary supplement is configured to be convenient in use and linked to notice accessible to public and at least referencing at least one aspect of partial autophagy.

11. The dietary supplement of claim 9, wherein the dietary supplement recommended daily dose of: the lutein is 1 milligram to 6 milligrams per day; the curcumin is 0.01 gram to 8 grams per day; the ornithine is 0.01 grams to 0.8 grams per day; the agmatine is 0.02 milligrams to 3 grams per day; the piperine is 0.1 milligram to 20 milligrams per day, the taurine is 10 milligrams to 3 grams per day, and green coffee bean extract is 5 milligrams to 500 milligrams per day.
